(19) United States
(12) Patent Application Publication
(10) Pub. No.: US 2026/0366071 A1
(43) Pub. Date: Nov. 19, 2026

(54) QUANTITATIVE ANALYSIS METHOD FOR GENE MUTATION, QUANTITATIVE ANALYSIS DEVICE, AND QUANTITATIVE ANALYSIS KIT

(71) Applicant: HITACHI, LTD., Tokyo (JP)

(72) Inventors: Takahiro ANDO, Tokyo (JP); Takashi ANAZAWA, Tokyo (JP); Takahide YOKOI, Tokyo (JP)

(21) Appl. No.: 19/491,691

(22) PCT Filed: Jan. 19, 2024
(86) PCT No.: PCT/JP2024/001464

§ 371 Date: Dec. 9, 2025

(30) Foreign Application Priority Data
Jul. 3, 2023 (JP) 2023-109354

(51) Int. Cl. C12Q 1/6869; C12N 15/10; C12Q 1/6876

(52) U.S. Cl. CPC C12Q 1/6869; C12N 15/101; C12Q 1/6876; C12Q 2600/156

(57) ABSTRACT

The present invention relates to a method for quantitative analysis of a gene mutation, particularly a gene mutation which exists at a low frequency, as well as to a quantitative analysis device and a quantitative analysis kit for analysis of a gene mutation based on such a method. In detail, the present invention provides a method for quantitative analysis of a gene mutation, comprising the steps of: (a) adding an internal standard of a constant concentration labeled with a first fluorescent dye to a test sample; (b) dividing or preparing the test sample according to the number of types of bases to be detected; (c) adding a primer for a single-base extension reaction for detection of a target nucleotide sequence to the test sample; (d) performing a single-base extension reaction using the divided or prepared test sample for each base type, the primer for a single-base extension reaction for detection of a target nucleotide sequence, and a terminator labeled with a second fluorescent dye corresponding to each base type; (e) subjecting a product of the single-base extension reaction to electrophoresis for each base type; and (f) measuring mobility in the electrophoresis and a fluorescence intensity of each of the fluorescent dyes, and detecting a wild type and a mutant of the target nucleotide sequence based on the fluorescence intensity, wherein a ratio of the fluorescence intensity of the second fluorescent dye in a peak of the product of the single-base extension reaction with respect to the fluorescence intensity of the first fluorescent dye in a peak of the internal standard obtained for each base type is calculated, and based on the ratio, a quantitative ratio of the product of the single-base extension reaction for each base type is quantified.

### TECHNICAL FIELD

The present invention relates to a method for quantitative analysis of a gene mutation, particularly a gene mutation which exists at a low frequency, as well as to a quantitative analysis device and a quantitative analysis kit for analysis of a gene mutation based on such a method.

### BACKGROUND ART

With the advancement of genetic analysis technologies, correlations between various diseases and gene mutations are being elucidated. Mutations of the somatic cell lineage, which are acquired gene mutations derived from a disease such as cancer, have the characteristics that the mutation sites on the genome cannot be predicted, and that the abundance ratio of mutation within an individual or tissue cannot be predicted. For example, a tissue sample of a tumor site excised from a patient with cancer contains both cancer cells and normal cells, and there is also diversity in gene mutations among the cancer cells themselves. Therefore, the abundance ratio of cells having a gene mutation at a specific position of a specific gene in a sample may be extremely low. Accordingly, a highly sensitive detection method is required in order to detect an acquired gene mutation derived from a disease. In addition, in selection of a treatment method or a therapeutic drug, not only the presence or absence of a gene mutation at a specific position of a target gene but also the abundance ratio of such a gene mutation may be used as an indicator. Therefore, in addition to the highly sensitive detection of a gene mutation, quantification of the abundance ratio of such a gene mutation is also required.

One example of the gene mutation is single nucleotide polymorphism (SNP). For example, Non Patent Literature 1 demonstrates that, by using a single-base extension reaction kit for the detection of an SNP, SNaPshot (registered trademark), and fragment analysis by capillary electrophoresis device, it is possible to detect 120 known gene mutations in 13 cancer-related genes, after performing a multiplex polymerase chain reaction (PCR) on formalin-fixed and paraffin-embedded tissue to selectively enrich a target gene sequence derived from a tumor. In the measurement of the KRAS G12D mutation in the human lung cancer cell line A427 and the EGFR T790M mutation in a lung adenocarcinoma cell line NCI-H1975, it is shown that the detection sensitivity of the mutant with respect to the wild type is approximately 3%. It should be noted that the detectable allele frequency in the single-base extension reaction using the SNaPshot is typically about 5%. In this regard, as described in Non Patent Literature 1, it is considered that, due to large variations in the fluorescence intensities of the dideoxynucleotide triphosphates (terminators) labeled with the fluorescent dye, that is, ddATP, ddCTP, ddGTP, and ddTTP, that act on the four bases: adenine (A), cytosine (C), guanine (G), and thymine (T), as well as to the presence of unnecessary peaks other than those of the products resulting from the single-base extension reaction, the detection sensitivity is limited to several percent.

From the viewpoint of improving the detection sensitivity of gene mutations, Patent Literature 1 reports on an approach to eliminate the influence of fluorescence signal crosstalk (a change in fluorescence signal intensity caused by interference between multiple fluorescent signals), which causes sensitivity reduction in the detection of a trace genetic polymorphism of the somatic cell lineage. A method has been proposed in which a nucleic acid sample is divided and reacted separately for each base type, subjected to electrophoresis for each base type in individual channels, and gene mutations are detected based on waveform data obtained from labeling signals specific to each base type for the nucleic acid samples electrophoresed in the respective channels (however, no specific degree of improvement in the detection sensitivity is described). Further, from the viewpoint of improving quantification accuracy, Patent Literature 2 proposes a method for obtaining information on trace gene mutations present in a target gene region with high sensitivity and accuracy by improving normalization accuracy. Specifically, nucleic acid samples of analysis targets labeled for each base type are subjected to electrophoresis, the labeling signals are detected to generate waveform data of detection intensity for each base type, other peak positions are selected for each peak position in the waveform data for each base type, the relative signal intensity of each peak position is calculated with respect to the signal intensity of the selected other peak position, and the relative signal intensity of the analysis target nucleic acid sample is compared with the relative signal intensity of a known nucleic acid sample at each peak position for analysis of each base type at nucleotide sequence coordinate positions of the nucleic acid sample.

Furthermore, when detecting a trace gene mutation, such as in a case where the abundance ratio of a mutant with respect to a wild type is less than 1%, an approach that simply increases the sample concentration can also be considered. In this case, the internal standard method is used to quantify mutant genes. In the internal standard method, it is required that the signal intensity for each of the analysis target and the internal standard be proportional to its concentration, that is, the increase in each signal intensity with respect to the concentration of the analysis target and the internal standard must be linear. Assuming that the linearity of the signal intensity is maintained when the sample concentration is increased, quantitative detection of gene mutations becomes possible based on such characteristics.

### CITATION LIST

### Patent Literature

Patent Literature 1: JP 5723993 B (US 2014/0336949 A1)
Patent Literature 2: JP 5789720 B (U.S. Pat. No. 10,274,459 B)

### Non Patent Literature

Non Patent Literature 1: Dias-Santagata, D. et al., EMBO Molecular Medicine, Vol. 2, pp. 146-158 (2010)

### SUMMARY OF INVENTION

### Technical Problem

Examples of a gene mutation detection technology include detection of a gene mutation (for example, an SNP) using a product of a single-base extension reaction analyzed by fragment analysis by capillary electrophoresis. However, the detection sensitivity of the mutant with respect to the wild type is generally limited to several percent. In particular, when products of the single-base extension reaction labeled with different fluorescent dyes for the four bases are electrophoresed in each capillary, in addition to the influence of the fluorescence signal crosstalk, errors in signal intensity also occur during color conversion (unmixing of the fluorescence signals) for the base type identification, which impairs the quantitativity. To accurately and quantitatively detect a gene mutation which exists at a low frequency of 1% or less, it is necessary to devise a method for acquiring fluorescence signals and a method for processing the data. In addition, in detecting a gene mutation at a sensitivity of 1% or less, it is necessary to detect the fluorescence signals derived from wild-type and mutant samples in a wider concentration range (dynamic range) than in conventional methods. However, in the actual capillary electrophoresis, when attempting to apply the internal standard method for quantitative detection, there is a problem in that although a sample at a low concentration shows a linear increase in the fluorescence signal intensity with respect to the concentration, the fluorescence signal intensity of a sample at a concentration above or equal to a certain concentration becomes saturated.

### Solution to Problem

As a result of extensive studies to solve the above problem, the inventors have found that the abundance ratio of a gene mutation can be quantified by, prior to the single-base extension reaction, mixing a fluorescently labeled marker of a known base length which serves as an internal standard with the sample, dividing the sample according to the number of types of the bases to be detected, performing electrophoresis in separate capillaries, and comparing the ratio of the fluorescence signal intensity of a peak of a product of the single-base extension reaction with respect to the fluorescence signal intensity of a peak of the internal standard obtained in each capillary. This means that, by comparing the ratio of the fluorescence signal intensity of the peak of the product of the single-base extension reaction with respect to the fluorescence signal intensity of the peak of the internal standard obtained in each capillary, the quantification can be achieved without saturation of the fluorescence signal intensity even for a sample at a concentration above or equal to a certain concentration, and thus provides a solution to the problem described above, which can be achieved by applying the internal standard method to samples of a high dynamic range.

Accordingly, in one aspect, the present invention provides a method for quantitative analysis of a gene mutation, comprising the steps of:
(a) adding an internal standard of a constant concentration labeled with a first fluorescent dye to a test sample;
(b) dividing or preparing the test sample according to the number of types of bases to be detected;
(c) adding a primer for a single-base extension reaction for detection of a target nucleotide sequence to the test sample;
(d) performing a single-base extension reaction using the divided or prepared test sample for each base type, the primer for a single-base extension reaction for detection of a target nucleotide sequence, and a terminator labeled with a second fluorescent dye corresponding to each base type;
(e) subjecting a product of the single-base extension reaction to electrophoresis for each base type; and
(f) measuring mobility in the electrophoresis and a fluorescence intensity of each of the fluorescent dyes, and detecting a wild type and a mutant of the target nucleotide sequence based on the fluorescence intensity,
wherein a ratio of the fluorescence intensity of the second fluorescent dye in a peak of the product of the single-base extension reaction with respect to the fluorescence intensity of the first fluorescent dye in a peak of the internal standard obtained for each base type is calculated, and based on the ratio, a quantitative ratio of the product of the single-base extension reaction for each base type is quantified.

In another aspect, the present invention provides a device for quantitative analysis of a gene mutation for implementing the method for quantitative analysis of a gene mutation, comprising:
a measurement unit configured to perform a single-base extension reaction, electrophoresis, and a measurement of a fluorescence signal;
a data analysis unit comprising a measurement data storage part configured to store measurement data obtained by the measurement unit, and a data processing device; and
a control unit,
wherein the measurement unit is configured to perform electrophoresis of a product of the single-base extension reaction for each type of bases to be detected, and measure the fluorescence signal obtained from each electrophoresis, and
wherein the control unit is configured to control the data analysis unit so as to calculate a ratio of a fluorescence intensity of a fluorescent dye in a peak of the product of the single-base extension reaction with respect to a fluorescence intensity of a fluorescent dye in a peak of an internal standard obtained for each base type.

In a further aspect, the present invention provides a kit for quantitative analysis of a gene mutation for implementing the method for quantitative analysis of a gene mutation, comprising:
an internal standard of a constant concentration labeled with a first fluorescent dye;
a primer for a single-base extension reaction for detection of a target nucleotide sequence; and
a terminator for a single-base extension reaction labeled with a second fluorescent dye,
wherein the terminator is comprised in each separate container for each base type so as to be used in the single-base extension reaction for the corresponding base type.

The present specification includes the content of the disclosure of Japanese Patent Application No. 2023-109354 filed on Jul. 3, 2023, which forms the basis of the priority of the present application.

### Advantageous Effects of Invention

According to the present invention, in the detection of a gene mutation (SNP) of a product of a single-base extension reaction using the fragment analysis by capillary electrophoresis, it is possible to quantitatively measure a gene mutation present at a low frequency of 1% or less, thereby improving the accuracy of disease diagnosis related to genetic abnormalities, such as cancer. Further, it is possible to quantitatively analyze a gene mutation for which the abundance ratio of the mutant is 1% or less merely by devising the analysis method mainly related to a reagent, without special processing of electrophoresis data or modification of hardware. Problems, configurations, and effects other than those described above will be apparent from the following description of embodiments.

### BRIEF DESCRIPTION OF DRAWINGS

FIG. 1 is an explanatory diagram of fragment analysis by capillary electrophoresis for quantitatively measuring an abundance ratio of a mutant with respect to a wild type of a target nucleotide sequence using a single-base extension reaction.

FIG. 2 is a schematic diagram illustrating electric field injection of a sample (measurement sample) in a capillary electrophoresis device.

FIG. 3 is an explanatory diagram of a method for quantitatively analyzing a gene mutation which exists at a low frequency with a high dynamic range in capillary electrophoresis using a single-base extension reaction.

FIG. 4 is an explanatory diagram of one example of an embodiment in which, when an amount of a template DNA (test sample) used for the single-base extension reaction is small, the template DNA is captured and collected with magnetic beads to prevent reduction in sensitivity.

FIG. 5 is a flowchart illustrating one example of the processing procedure in a quantitative analysis device and a quantitative analysis kit for analyzing a gene mutation which exists at a low frequency for implementing the present invention.

FIG. 6 is a block diagram illustrating one example of a function included in the quantitative analysis device for analyzing a gene mutation according to the present invention.

FIG. 7 is a diagram illustrating one example of a user interface screen outputted by the quantitative analysis device for analyzing a gene mutation according to the present invention.

### DESCRIPTION OF EMBODIMENTS

Hereinafter, one example of an embodiment of the present invention will be described with reference to the drawings.

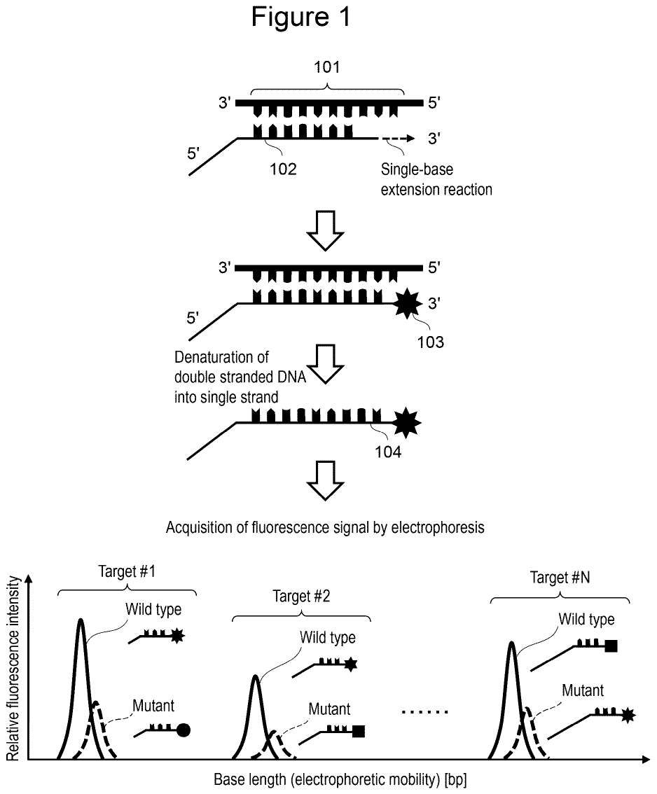

FIG. 1 is an explanatory diagram of fragment analysis by capillary electrophoresis for quantitatively measuring an abundance ratio of a mutant with respect to a wild type of a target nucleotide sequence using a single-base extension reaction. Generally, the analysis is designed so that a target nucleotide sequence 101 as a template DNA, and primers 102 having different base lengths (molecular weights) according to the target are used to perform a single-base extension reaction and the electrophoretic mobility of the products of the reaction is varied. By polymerase chain reaction (PCR), a ddNTP modified with one of four fluorescent dyes 103 corresponding to the four terminal bases, adenine (A), cytosine (C), guanine (G), and thymine (T), is added at the 3′ end position of the primer 102 corresponding to a gene mutation by the single-base extension reaction. By fluorescence detection of the fluorescent dye label 103 at the 3′ end of the product of the single-base extension reaction 104, in which the double-stranded DNA has been denatured into a single strand by a formamide treatment and heat denaturation, the gene mutation is identified. In such a case, by using primers having different base lengths according to the target, the electrophoretic mobility of each product of the single-base extension reaction can be varied.

It has been demonstrated in previous academic studies that, by using such primers having different base lengths according to the target, 20 or more types of known gene mutations can be detected in a single electrophoresis. Furthermore, by using the present technique, not only can a single nucleotide polymorphism (SNP), in which only one base is mutated, be detected, but a gene sequence of a specific length can also be detected depending on the primer design. Moreover, insertions (IN; Insertion) and deletions (DEL; Deletion), which are types of gene mutation, can also be detected based on the same principle.

Examples of the causes of the decrease in the detection sensitivity and quantitativity in the fluorescence detection include the influence of fluorescence signal crosstalk between adjacent products of the single-base extension reaction when they are electrophoresed in the capillaries, because the products of the single-base extension reaction which are labeled with different fluorescent dyes corresponding to the four bases are electrophoresed in the respective capillaries (that is, respectively in one capillary). Furthermore, errors in the fluorescence signal intensity also occur during color conversion (unmixing of the fluorescence signals) for identification of the base type, which impairs the quantitativity. Accordingly, software for analyzing the electrophoresis data is prepared to reduce the fluorescence signal crosstalk and the color conversion errors. It should be noted that a factor causing the color conversion errors is overlap of fluorescence spectra of the fluorescent dye labels such as X-rhodamine (ROX), rhodamine 110 (R110), rhodamine 6G (R6G), tetramethylrhodamine (TAMRA), fluorescein, or derivatives thereof like fluorescein isothiocyanate (FITC), Alexa 488, Alexa 532, Cy3, Cy5, and Texas Red. When the excitation wavelength of laser light installed in the capillary electrophoresis device is limited, it is technically difficult to completely separate (eliminate the overlap of) the fluorescence spectra of fluorescent dye labels used for the four types of bases. In addition to the fluorescent dye labels used for the four bases, in order to correct the electrophoretic mobility and the base length, a size standard bound to another fluorescent dye label is used as an internal standard. In this way, by mixing the internal standard at a constant concentration, the electrophoretic mobility and the base length are corrected, and the quantification of the products of the single-base extension reaction can be performed. However, in actual capillary electrophoresis, when attempting to apply this internal standard method for the quantitative detection, it has been confirmed that, while the fluorescence signal intensity increases linearly with respect to the concentration for a sample at a low concentration, the fluorescence signal intensity becomes saturated for a sample at a concentration above or equal to a certain concentration. The cause of this phenomenon will be explained in the next section.

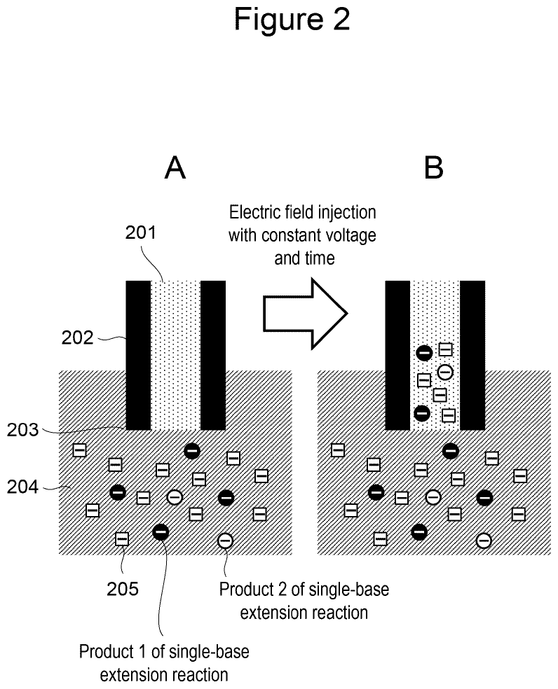

FIG. 2 is a schematic diagram illustrating electric field injection of a sample (measurement sample) in a capillary electrophoresis device. One end of a capillary 202 charged with a polymer solution 201 is immersed in a sample solution, and FIG. 2, A shows the state before electric field injection, whereas FIG. 2, B shows the state after the electric field injection. Although in the following description examples are given for products of the single-base extension reaction obtained for two types of target nucleotide sequences, the same considerations can be applied to products of the single-base extension reaction for three or more types of target nucleotide sequences.

In the state shown in FIG. 2, A, when the electric field injection is performed for a constant time T while a sample injection port 203 of the capillary is the cathode side and the sample elution port is the anode side, the state shown in FIG. 2, B is made. A current I which flows between the cathode and the sample injection port 203 of the capillary, that is, the current I which flows through the sample (measurement sample) 204, is carried by negative ions injected into the capillary, when positive ions are ignored. Here, when an effective electric field strength in the sample near the sample injection port of the capillary is E, and a cross-sectional area of the inner diameter of the capillary is A, the number Jᵢ of molecules injected into the capillary (products of the single-base extension reaction) is expressed by the following (Formula 1), using uᵢ and Cᵢ as the average mobility and concentration, respectively.

[Expression 1]

Jᵢ=E·T·A·μᵢ·Cᵢ (Formula 1)

Here, it is assumed that a product 1 of the single-base extension reaction corresponding to a certain target nucleotide sequence 1 corresponds to i=1, and a product 2 of the single-base extension reaction corresponding to another target nucleotide sequence 2 corresponds to i=2. The mobility indicates the migration speed per unit electric field strength for each negative ion. When an average charge amount of each molecule is qᵢ, a charge amount Qᵢ injected into the capillary is expressed by the following (Formula 2).

[Expression 2]

Qᵢ=qᵢ·Jᵢ=E·T·A·qᵢ·μᵢ·Cᵢ (Formula 2)

In this case, based on the relationship I·T=ΣᵢQᵢ, when the mobility of negative ions 205 other than the products of the single-base extension reaction in the sample is represented as i=0, the following (Formula 3) is obtained.

$$[ Expression 3 ]$$

$$E \cdot A = \frac{I}{{q}_{0} \cdot {μ}_{0} \cdot {C}_{0} + {\sum }_{i} {q}_{i} \cdot {μ}_{i} \cdot {C}_{i}} \qquad ( Formula 3 )$$

When the average charge amount q and the average mobility μ of each product of the single-base extension reaction are equal and a total concentration is assumed to be C, a total number J of the molecules injected into the capillary and a total charge amount Q are expressed by the following (Formula 4) and (Formula 5), respectively, based on the (Formula 1), the (Formula 2), and the (Formula 3).

$$[ Expression 4 ]$$

$$J = \frac{\frac{I \cdot T}{q}}{1 + \frac{{q}_{0} \cdot {μ}_{0} \cdot {C}_{0}}{q \cdot μ \cdot C}} \qquad ( Formula 4 )$$

$$Q = \frac{I \cdot T}{1 + \frac{{q}_{0} \cdot {μ}_{0} \cdot {C}_{0}}{q \cdot μ \cdot C}} \qquad ( Formula 5 )$$

Furthermore, a fluorescence signal intensity Sᵢ of the peak of each product of the single-base extension reaction obtained from the fragment analysis by the capillary electrophoresis on electropherogram is represented by the following (Formula 6) using a sensitivity coefficient mᵢ.

[Expression 5]

Sᵢ=mᵢ·Jᵢ=E·T·A·mᵢ·μᵢ·Cᵢ (Formula 6)

It should be noted that the sensitivity coefficient mᵢ includes factors such as the average number of fluorescent dye labels per product of the single-base extension reaction, an excitation efficiency of the fluorophore, a quantum yield of the fluorophore, a light collection efficiency of the emitted fluorescence, and sensitivity of the image sensor. Using the (Formula 6), the total fluorescence signal intensity of the peaks are expressed by the following (Formula 7), when the sensitivity coefficient is assumed to be equally m.

$$[ Expression 6 ]$$

$$S = \frac{\frac{m \cdot I \cdot T}{q}}{1 + \frac{{q}_{0} \cdot {μ}_{0} \cdot {C}_{0}}{q \cdot μ \cdot C}} \qquad ( Formula 7 )$$

This Formula (7) is the very reason why, when the internal standard method is applied for the quantitative detection in the actual capillary electrophoresis, the fluorescence signal intensity increases linearly with respect to the concentration for a sample at a low concentration but becomes saturated for a sample at a concentration above or equal to a certain concentration (when C becomes excessively large). Considering the relationship for each product of the single-base extension reaction, it can be expressed by the following (Formula 8), based on the (Formula 6) and the (Formula 7).

$$[ Expression 7 ]$$

$$\frac{{S}_{i}}{S} = \frac{{C}_{i}}{C} \qquad ( Formula 8 )$$

That is, a ratio of the fluorescence signal intensity Sᵢ of each product of the single-base extension reaction with respect to the total fluorescence signal intensity S of the products of the single-base extension reaction injected into the capillary by the electric field injection corresponds to a ratio of the concentration Cᵢ of each product of the single-base extension reaction with respect to the total concentration C of the products of the single-base extension reaction in the sample. At the same time, based on the (Formula 8), for example, for the product 1 of the single-base extension reaction corresponding to the certain target nucleotide sequence 1 and the product 2 of the single-base extension reaction corresponding to another target nucleotide sequence 2, the following (Formula 9) is derived.

$$[ Expression 8 ]$$

$$\frac{{S}_{2}}{{S}_{1}} = \frac{{C}_{2}}{{C}_{1}} \qquad ( Formula 9 )$$

By using this relational expression, when comparing the ratio of the fluorescence signal intensity of the peak of each product of the single-base extension reaction with respect to the fluorescence signal intensity of the peak of the internal standard obtained in each capillary, the quantification can be achieved with a high dynamic range without the saturation of the fluorescence signal intensity even for a sample of a certain concentration or higher.

According to the present invention, a gene mutation to be detected may be quantitatively detected with high sensitivity and accuracy by adding the internal standard and performing the electrophoresis separately for each type of base to be detected in a gene analysis method combining a single-base extension reaction and electrophoresis. Accordingly, in one aspect, the present invention relates to a method for quantitative analysis of a gene mutation, comprising the steps of:
(a) adding an internal standard of a constant concentration labeled with a first fluorescent dye to a test sample;
(b) dividing or preparing the test sample according to the number of types of bases to be detected;
(c) adding a primer for a single-base extension reaction for detection of a target nucleotide sequence to the test sample;
(d) performing a single-base extension reaction using the divided or prepared test sample for each base type, the primer for a single-base extension reaction for detection of a target nucleotide sequence, and a terminator labeled with a second fluorescent dye corresponding to each base type;
(e) subjecting a product of the single-base extension reaction to electrophoresis for each base type; and
(f) measuring mobility in the electrophoresis and a fluorescence intensity of each of the fluorescent dyes, and detecting a wild type and a mutant of the target nucleotide sequence based on the fluorescence intensity,
wherein a ratio of the fluorescence intensity of the second fluorescent dye in a peak of the product of the single-base extension reaction with respect to the fluorescence intensity of the first fluorescent dye in a peak of the internal standard obtained for each base type is calculated, and based on the ratio, a quantitative ratio of the product of the single-base extension reaction for each base type is quantified.

According to the present invention, the term “target nucleotide sequence” refers to a nucleotide sequence for which the detection and the quantification are desired. For example, the target nucleotide sequence may be a nucleotide sequence including a gene mutation, and the target nucleotide sequence may be detected (and quantified) by distinguishing between the wild-type and mutant sequences. Examples of such a gene mutation include, but are not limited to, single nucleotide polymorphisms (SNPs), insertion mutations (IN: Insertion), deletion mutations (DEL: Deletion), microsatellite repeats, copy number variations (CNVs), and epigenetic mutations. The mutant of the target nucleotide sequence may include either one mutant or a plurality of mutants. For example, there may be a gene mutation having two or three mutant types in addition to the wild type, and such multiple mutants can also be distinguished, detected, and quantified according to the present invention.

The test sample subjected to the present method is not particularly limited as long as it is a nucleic acid sample intended for detection of the target nucleotide sequence, and examples of such a sample include deoxyribonucleic acids (DNAs), such as a genomic DNA or a cDNA, and ribonucleic acids (RNAs), such as a messenger RNA (mRNA), as well as fragments thereof. According to the present invention, it is preferable to use, as the test sample, for example, a cell-free DNA (cfDNA, DNA freely circulating in the bloodstream) or a circulating tumor DNA (ctDNA). Preparation of the nucleic acid from the sample can be carried out by a method known in the art. Kits for nucleic acid preparation are commercially available from many manufacturers, and thus it is possible to easily purify the desired nucleic acid.

Hereinafter, each embodiment of the method for quantitative analysis of a gene mutation according to the present invention will be described.

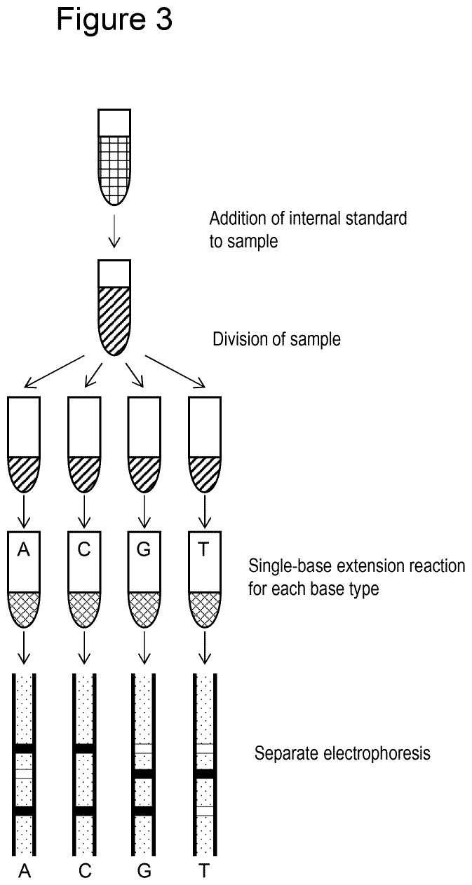

FIG. 3 is an explanatory diagram of one example of a method for quantitatively analyzing a gene mutation which exists at a low frequency with a high dynamic range in capillary electrophoresis using a single-base extension reaction. In one embodiment of the present invention, an internal standard of a constant concentration may be first added to a sample (test sample) in which wild-type and mutant sequences of a target nucleotide sequence coexist. As the internal standard, one or more fluorescently labeled DNA fragments of known or fixed concentration, a size standard bound to a fluorescent dye label for correcting the electrophoretic mobility and the base length, and the like may be used. Next, the sample may be divided according to the number of types of the bases to be detected. Although FIG. 3 shows an example of division corresponding to four types of bases, if two or three types of bases are to be detected, the sample may be divided into two or three portions. When an internal standard which is unaffected (or only slightly affected) by the single-base extension reaction, such as a size standard, is used, adding first the internal standard enables to reduce dispensing errors that would otherwise occur when adding the internal standard after the division. Thus, the final concentration of the internal standard can be kept constant and the quantification accuracy can be improved. The divided samples may then be subjected to a single-base extension reaction using a terminator labeled with one type of fluorescent dye, for each base type.

When a substance affected by the single-base extension reaction is used as the internal standard, for example, the product of the single-base extension reaction at an SNP site (gene mutation site) having the same base type as the detection target and a known abundance ratio (for example, 100%) may be used. In this case, the minimum number of capillaries required is one. For example, by a single capillary electrophoresis, the abundance ratio of the base A at an SNP site 2 can be quantified based on the ratio of the peak fluorescence intensity of the product of the single-base extension reaction at the SNP site 2 (where the ratio of the base A is unknown) with respect to the peak fluorescence intensity of the product of the single-base extension reaction at an SNP site 1 (where the ratio of the base A is 100%).

When four capillaries are used, the internal standards may similarly be required for at least the four types of bases A, C, G, and T. When the single-base extension reaction is performed for each base type, not only terminators labeled with fluorescent dyes corresponding to the target base types but also terminators without a fluorescent label corresponding to the remaining three, non-target bases may be mixed to suppress errors such as nonspecific adsorption during reagent reactions.

The incorporation efficiency of the terminator may vary depending on the mutation site (SNP site), base type, and fluorescent dye label bound to the terminator. The excitation efficiency and the quantum yield may differ depending on the type of fluorophore used for the fluorescent dye label. If the comprehensive efficiency is defined as a sensitivity coefficient K (m, n) (where m represents the mutation (SNP) number and n represents the base type), K (m, n) can be determined based on a slope of a calibration curve which is obtained in advance for each mutation (SNP) number and base type. For example, for the four types of bases A, C, G, and T, when the fluorescence intensity peak of the internal standard obtained in the capillary for the base A is Sₐ, the fluorescence intensity peak of the product of the single-base extension reaction obtained in the capillary for the base A is Tₐ, the fluorescence intensity peak of the internal standard obtained in the capillary for the base C is Se, the fluorescence intensity peak of the product of the single-base extension reaction obtained in the capillary for the base C is T_{c}, the fluorescence intensity peak of the internal standard obtained in the capillary for the base G is S_{g}, the fluorescence intensity peak of the product of the single-base extension reaction obtained in the capillary for the base G is T_{g}, the fluorescence intensity peak of the internal standard obtained in the capillary for the base T is Sₜ, and the fluorescence intensity peak of the product of the single-base extension reaction obtained in the capillary for the base T is Tₜ, the abundance ratio of A, C, G, and T can be quantified as Tₐ/Sₐ, T_{c}d/S_{c}, T_{g}/S_{g}, and Tₜ/Sₜ, while preventing the saturation of the fluorescence signal intensity. Moreover, in addition to the simple quantification as Tₐ/Sₐ, T_{c}/S_{c}, T_{g}/S_{g}, and Tₜ/Sₜ, it is preferable to perform quantification by multiplication of the sensitivity coefficients, that is, K (m, a)×Tₐ/Sₐ, K (m, c)×T_{c}/S_{c}, K (m, g)×T_{g}/S_{g}, and K (m, t)×Tₜ/Sₜ. Furthermore, it is also possible to design the primers for the single-base extension reaction so that the fluorescence signal peak of the internal standard and the peak of the product of the single-base extension reaction of the target nucleotide sequence do not overlap on the electropherogram. In such a case, the same fluorescent dye label may be bound to the product of the single-base extension reaction and the internal standard.

Accordingly, in one embodiment, the steps described above may be carried out in the order of the steps (a), (b), (c), (d), (e), and (f).

In detail, the method for quantitative analysis of a gene mutation according to the present invention relates to a method for quantitative analysis of a gene mutation, comprising the steps of
(a) adding an internal standard of a constant concentration labeled with a first fluorescent dye to a test sample;
(b) dividing or preparing the test sample according to the number of types of bases to be detected;
(c) adding a primer for a single-base extension reaction for detection of a target nucleotide sequence to the test sample;
(d) performing a single-base extension reaction using the divided or prepared test sample for each base type, the primer for a single-base extension reaction for detection of a target nucleotide sequence, and a terminator labeled with a second fluorescent dye corresponding to each base type;
(e) subjecting a product of the single-base extension reaction to electrophoresis for each base type; and
(f) measuring mobility in the electrophoresis and a fluorescence intensity of each of the fluorescent dyes, and detecting a wild type and a mutant of the target nucleotide sequence based on the fluorescence intensity,
wherein a ratio of the fluorescence intensity of the second fluorescent dye in a peak of the product of the single-base extension reaction with respect to the fluorescence intensity of the first fluorescent dye in a peak of the internal standard obtained for each base type is calculated, and based on the ratio, a quantitative ratio of the product of the single-base extension reaction for each base type is quantified.

In another embodiment, the steps described above may be carried out in the order of the steps (c), (a), (b), (d), (e), and (f). In detail, the method for quantitative analysis of a gene mutation according to the present invention relates to a method for quantitative analysis of a gene mutation, comprising the steps of
(c) adding a primer for a single-base extension reaction for detection of a target nucleotide sequence to the test sample;
(a) adding an internal standard of a constant concentration labeled with a first fluorescent dye to the test sample;
(b) dividing or preparing the test sample according to the number of types of bases to be detected;
(d) performing a single-base extension reaction using the divided or prepared test sample for each base type, the primer for a single-base extension reaction for detection of a target nucleotide sequence, and a terminator labeled with a second fluorescent dye corresponding to each base type;
(e) subjecting a product of the single-base extension reaction to electrophoresis for each base type; and
(f) measuring mobility in the electrophoresis and a fluorescence intensity of each of the fluorescent dyes, and detecting a wild type and a mutant of the target nucleotide sequence based on the fluorescence intensity,
wherein a ratio of the fluorescence intensity of the second fluorescent dye in a peak of the product of the single-base extension reaction with respect to the fluorescence intensity of the first fluorescent dye in a peak of the internal standard obtained for each base type is calculated, and based on the ratio, a quantitative ratio of the product of the single-base extension reaction for each base type is quantified.

In the step (a), an internal standard of a constant concentration labeled with a first fluorescent dye may be added to a test sample. As the internal standard, a DNA fragment of a known or fixed concentration, a size standard for correcting the electrophoretic mobility and the base length, or the like may be used. Alternatively, as the internal standard, a product of a single-base extension reaction of a known concentration that has been extended with the same base type as the base to be detected may be used. The first fluorescent dye is useful for detecting the abundance or the proportion of the presence of the internal standard in the subsequent analysis, and fluorescent dyes known in the art can be used. Examples of the fluorescent dye include, but are not limited to, fluorescein, fluorescein isothiocyanate (FITC), sulforhodamine (TR), tetramethylrhodamine (TRITC), carboxy-X-rhodamine (ROX), carboxytetramethylrhodamine (TAMRA), NED, 5-carboxyfluorescein (5-FAM), 6-carboxyfluorescein (6-FAM), 5′-hexachlorofluorescein CE-phosphoramidite (HEX), 6-carboxy-4′,5′-dichloro-2′,7′-dimethoxyfluorescein (JOE), 5′-tetrachlorofluorescein CE-phosphoramidite (TET), rhodamine 110 (R110), rhodamine 6G (R6G), VIC (registered trademark), ATTO type dyes, Alexa Fluor (registered trademark) type dies, Texas Red, Cy type dyes, and the like. Further, examples of the fluorescent dyes that do not cause shifts in electrophoretic size include dR110 (carboxy-dichloro rhodamine 110), dR6G (dihydro rhodamine 6G), dTAMRA (tetramethylrhodamine), dROX (carboxy-X-rhodamine), and the like. The type of the fluorescent dye and the method for the introduction of the fluorescent dye are not particularly limited, and various conventional techniques known in the art may be employed.

The addition of the internal standard may be performed at the beginning of the method, before the single-base extension reaction is performed, or before the electrophoresis is performed.

In the step (b), the test sample may be divided or prepared according to the number of types of bases to be detected. The types of bases to be detected correspond to the types of gene mutations (wild type and mutant) to be detected. For example, when one type of target nucleotide sequence is to be detected, in which only one mutant exists in addition to the wild type, the number of the types of bases to be detected is two, and the test sample may be divided into two portions or prepared in duplicate (for example, two test samples are prepared by amplification). When two or more mutants exist or when multiple target nucleotide sequences are to be detected, the number of the types of bases to be detected is three or four. In one embodiment, the test sample may be divided into two, three, or four portions. It is preferable to divide (equally) or prepare the test sample in equal volumes, but the equal division is not necessary when the division is performed after the addition of the internal standard.

The step (c) to the step (e) can be carried out using a gene analysis method combining a single-base extension reaction and electrophoresis, and such a gene analysis method is well known in the art, for example, as described in Non-Patent Literature 1.

The single-base extension reaction may be carried out using, as a substrate, a terminator (dideoxynucleotide triphosphates: ddNTPs) labeled with a fluorescent dye, and a primer for a single-base extension reaction for detection of a target nucleotide sequence. In the step (c), the primer for single-base extension reaction for detection of a target nucleotide sequence may be added.

The primer for a single-base extension reaction may be either a DNA or an RNA, and may be determined according to the type of the test sample, the type of the target nucleotide sequence, and the type of the polymerase used in the single-base extension reaction. Preferably, the primer is a DNA, and the single-base extension reaction is performed using a DNA or an mRNA as the template in the test sample.

Each primer may be designed such that at least a portion (on the 3′ end side) has a sequence specific to the target nucleotide sequence, that is, a sequence complementary to the target nucleotide sequence. The base length of the primer affects the base length (size) of the product of the single-base extension reaction, and thus the electrophoretic mobility may be determined based on the base length of the primer. Therefore, in a gene analysis method combining a single-base extension reaction and electrophoresis, it is necessary to appropriately design the nucleotide sequence and the base length of the primer. For example, when attempting to detect multiple (for example, two or more) target nucleotide sequences, for each target nucleotide sequence, a primer having a sequence portion specific to the target nucleotide sequence and a different base length that produces a difference in the electrophoretic mobility is designed. The portion that produces a difference in the base length does not need to be specific to the target nucleotide sequence (for example, the 5′ region of the primer shown in FIG. 1). Since a primer is required for each target nucleotide sequence, the number of primers designed corresponds to the type of target nucleotide sequences to be detected. For example, in order to detect five or more (for example, 5 to 100) target nucleotide sequences, preferably ten or more (for example, 10 to 100) target nucleotide sequences, different primers corresponding to the respective target nucleotide sequences may be used.

Methods for designing primers are well known in the art. The primers that can be used in the present invention may be designed to meet conditions that enables specific annealing, for example, by having a length and a base composition (a melting temperature) that enable the specific annealing. For example, the length for serving as the primer is preferably a length of 10 bases or more, more preferably 15 to 50 bases, and even more preferably 15 to 30 bases, for example, 20 bases. Further, in the designing, it is preferable to confirm the GC content and the melting temperature (Tm) of the primer. Known primer designing software can be used for confirming the Tm. The designed primers can be chemically synthesized by a known oligonucleotide synthesis technique, and are generally synthesized using a commercially available chemical synthesis device.

In addition to having the sequence portion of the different base length as described above for adjusting the electrophoretic mobility, the primer may also have a tag (a molecular weight), such as an interstrand-crosslinked double-stranded DNA tag, to provide a different mobility. The present inventors have previously developed a fragment analysis method using capillary electrophoresis, and developed an analysis method capable of expanding the number of gene mutations that can be simultaneously detected to several tens to several hundreds of types. In detail, by linking an interstrand-crosslinked double-stranded DNA tag to a primer and using the primer, the electrophoretic distance can be stably extended to 120 bp or more by changing the length of the double-stranded DNA tag to extend the base length of the primer, thereby making it possible to increase the number of gene mutations which can be simultaneously detected. The double-stranded DNA tag has a length distinguishable by the mobility and contains at least one interstrand crosslink. According to the present invention, the term “interstrand crosslink” means that one strand and the other strand of the double-stranded DNA are crosslinked at least one site. The method for forming an intramolecular crosslink between such two strands is not particularly limited as long as it is a method known in the art. Preferably, the interstrand crosslink is formed by photocrosslinking. The double-stranded DNA tag having the interstrand crosslink defines the migration distance (the mobility) in electrophoresis. That is, by linking the primer with the double-stranded DNA tag of a different length, the migration distance in the electrophoresis can be varied. Since capillary electrophoresis can detect nucleic acids with base lengths up to about 600 bases, excluding the base length (10 to 30 bases) of the primer region binding to the target nucleotide sequence, the double-stranded DNA tag may have a length within the range of 1 to about 590 bases. The nucleotide sequence of the double-stranded DNA tag is not particularly limited as long as it is a nucleic acid having an interstrand crosslink. Further, the double-stranded DNA tag can be chemically synthesized by a known oligonucleotide synthesis method, and is generally synthesized using a commercially available chemical synthesis device.

In this case, when the multiple target nucleotide sequences include a target nucleotide sequence for which more quantitative gene analysis is desired, it is preferable to assign the target nucleotide sequence item requiring higher quantitativity to a base length region where higher resolution and accuracy are exhibited in the capillary electrophoresis device (generally set to be around 50 to 100 bp, although it varies depending on the specification of the device). Due to the nature of the fragment analysis, the electrophoretic resolution tends to decrease as the base length increases, and therefore, by using a short-base primer for the target nucleotide sequence requiring more accurate quantitativity, the detection precision can be improved.

In the step (d), the single-base extension reaction may be performed using the divided or prepared test sample for each base type, the primer for a single-base extension reaction for detection of a target nucleotide sequence, and a terminator labeled with a second fluorescent dye corresponding to each base type. When performing the single-base extension reaction, it is preferable to divide or prepare the test sample for each base type to be detected, and to perform the single-base extension reaction on each divided or prepared test sample.

The single-base extension reaction is known in the art and is typically a single-base extension reaction using a polymerase. The polymerase used may be selected depending on the type of the template (the test sample) and the type of the primer used. For example, in a single-base extension reaction using a DNA primer with a DNA or RNA template, a DNA-dependent or RNA-dependent DNA polymerase is used, respectively.

The single-base extension reaction is widely known in the art, and, for example, Non-Patent Literature 1 describes a method for efficiently extending one base by a cycle reaction, and the like.

When a target nucleotide sequence is present, the primer hybridizes to this target nucleotide sequence, and a nucleotide is incorporated as a substrate through a polymerase synthesis reaction starting from the 3′ end portion of the primer. At this time, by using a dideoxynucleotide (ddNTP) as the nucleotide (substrate) to be incorporated, the synthesis reaction terminates after the extension by a single base. Therefore, this ddNTP is also referred to as a terminator.

In the step (d), the terminator labeled with a second fluorescent dye may be used as the substrate for the single-base extension reaction. Here, the terminator includes two or more types of terminators labeled with different types of second fluorescent dyes that can distinguish between the types of bases corresponding to the bases to be detected. The second fluorescent dye is useful for easily detecting whether or not the substrate has been incorporated or for determining the type of the incorporated base, and fluorescent dyes known in the art may be used. As the second fluorescent dye, those exemplified as the first fluorescent dye may be used, and the first fluorescent dye and the second fluorescent dye may be of either the same or different types. Preferably, the first fluorescent dye and the second fluorescent dye are fluorescent dyes of different types. For example, when attempting to determine the type of the base, five fluorescent dyes excited and detected at different wavelengths may be used in combination to distinguish four types of bases and one reference (for detecting and correcting the base length from a reference ladder DNA).

In one embodiment, in the step (d), the single-base extension reaction may be performed in the presence of, in addition to the terminator labeled with the second fluorescent dye corresponding to each base type, a terminator unlabeled with a fluorescent dye corresponding to a base type different from the base type. This can suppress an error such as nonspecific adsorption during the reagent reaction.

After the single-base extension reaction, in the step (e), the product of the single-base extension reaction may be subjected to electrophoresis for each base type. The electrophoresis is not particularly limited as long as it is a measurement method that enables the fragment analysis by electrophoresis. For example, electrophoresis in microchannels, such as capillary electrophoresis (CE) and MEMS (Micro-Electro-Mechanical Systems), may be used. In a preferred embodiment, the electrophoresis is the capillary electrophoresis (CE).

The electrophoresis, such as the CE, is a technique that separates introduced components based on differences in mobility determined by the charge, the size, the shape, and the like. Based on the mobility, the type of the target nucleotide sequence (according to the type of the primer) can be identified. Further, based on the signal from the fluorescent dye, it is possible to determine the presence or absence of the target nucleotide sequence, or the specific type of base in the target nucleotide sequence (based on the type of the substrate incorporated during the single-base extension reaction), for example, wild type or mutant.

In the step (f), the electrophoretic mobility and the fluorescence intensity of the fluorescent dye may be measured, and the wild type and the mutant of the target nucleotide sequence may be detected based on the fluorescence intensity. At that time, the ratio of the fluorescence intensity of the second fluorescent dye in the peak of the product of the single-base extension reaction with respect to the fluorescence intensity of the first fluorescent dye in the peak of the internal standard obtained for each base type may be calculated, and based on the ratio, it is possible to determine the quantitative ratio of the product of the single-base extension reaction for each base type. That is, it is possible to quantitatively detect the wild type and the mutant of the target nucleotide sequence.

As described above, the ratio Tᵢ/Sᵢ, that is, the ratio of the fluorescence signal intensity Tᵢ in the peak of the product of the single-base extension reaction for each base type with respect to the fluorescence signal intensity Sᵢ in the peak of the internal standard obtained in each capillary, that is, obtained for each base type, may be determined to quantify the quantitative ratio of the product of the single-base extension reaction for each base type. The conventional internal standard method was applicable only when the signal intensity is proportional to the concentration of the analysis target. In contrast, the internal standard method used in the present invention can be applied not only when the signal intensity is proportional to the concentration of the analysis target, but also when it is not proportional, when it is approaching saturation, or even when it reaches the saturation. By plotting the relative fluorescence intensity in this manner, it is possible to quantify the products of the single-base extension reaction (gene mutations) using the internal standard method with high accuracy over a wider concentration range of the target than in a conventional internal standard method.

In one embodiment, when in the step (a) the internal standard of a constant concentration may be a product of a single-base extension reaction of a known concentration extended by a base of the same base type as a base to be detected, the ratio of the fluorescence intensity of the second fluorescent dye in the peak of the product of the single-base extension reaction with respect to the fluorescence intensity of the first fluorescent dye in the peak of the product of the single-base extension reaction of the known concentration may be calculated, and based on the ratio, the quantitative ratio of the product of the single-base extension reaction for each base type may be determined.

By quantitatively detecting the wild type and the mutant of the target nucleotide sequence, it is possible to quantify, for example, the abundance ratio of the mutant sequence with respect to the wild-type sequence, which is required for cancer diagnosis, or the frequency of gene mutations. In one embodiment, a plurality of target nucleotide sequences to be analyzed include the wild-type sequence and the mutant sequence, and analysis of the target nucleotide sequences can be performed when the content ratio of the mutant sequence with respect to the wild-type sequence is in the range of 0.01% to 10%, for example, in the range of 0.01% to 1%, or in the range of 0.01% to 0.1%. In this way, the quantitative gene analysis of the target nucleotide sequence can be performed.

In another embodiment, the method of the present invention further comprises, after performing the steps (c) and (d), a step of:
(g) capturing the test sample that serves as a template for the single-base extension reaction using a solid phase for capturing the test sample and collecting a reaction solution comprising the product of the single-base extension reaction,
wherein the steps (c), (d), and (g) are repeatedly carried out the number of times corresponding to the number of types of bases to be detected using the same test sample to obtain, as the step (b), a product of a single-base extension reaction prepared for each type of base to be detected corresponding to the test sample, and thereafter the steps (a), (e), and (f) are carried out. In this embodiment, by repeatedly performing the single-base extension reaction the number of times corresponding to the number of types of the bases to be detected using the test sample as a template, a product of the single-base extension reaction corresponding to the test sample may be prepared for each type of base to be detected.

In the step (g), the test sample may be captured using a solid phase. The solid phase is not particularly limited as long as it can capture the test sample, and a commonly used solid phase in the field of gene analysis can be employed, such as a solid phase to which a probe is immobilized, for example, beads (preferably magnetic beads), a solid surface, or fibers. The immobilized probe may vary depending on the type of the test sample, and a person skilled in the art can design and synthesize an appropriate probe and immobilize it on the solid phase. After capturing the test sample, the reaction solution containing the residual product of the single-base extension reaction may be collected and subjected to subsequent steps. The captured test sample may be reused as a template for the single-base extension reaction for another base type to be detected. By repeatedly performing the single-base extension reaction and the test sample capture of the steps (c), (d), and (g), the product of the single-base extension reaction corresponding to the teste sample may be prepared for each base to be detected (corresponding to the step (b)).

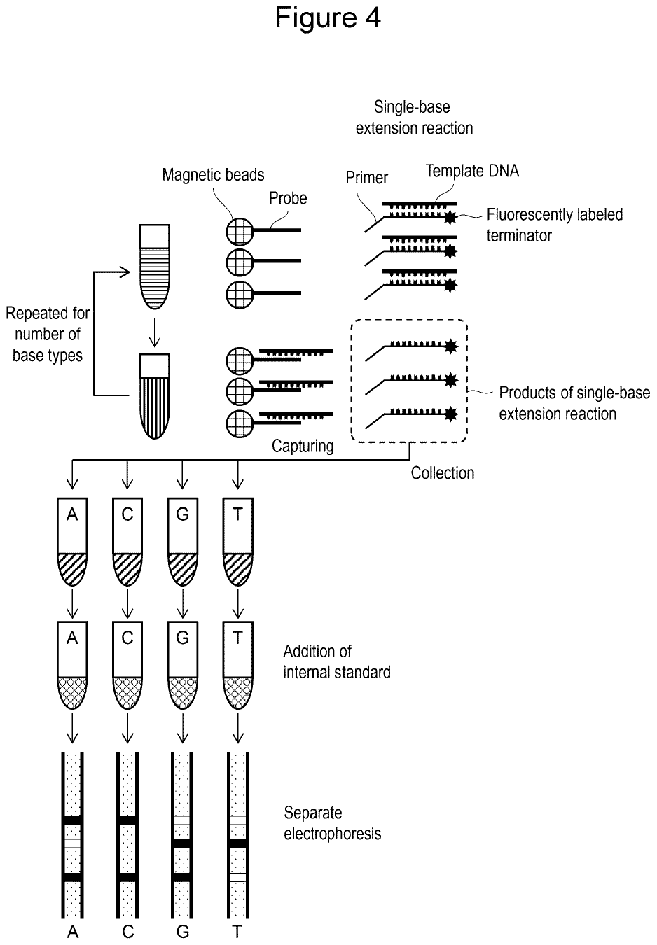

FIG. 4 is an explanatory diagram of one example of an embodiment in which, when an amount of a template DNA (test sample) used for the single-base extension reaction is small, the template DNA is captured and collected with magnetic beads to prevent reduction in sensitivity. When the target nucleotide sequence in the test sample serves as the template DNA, even if the DNA is amplified by PCR or the like before the single-base extension reaction, there may be a case in which the amount of the mutant template DNA becomes particularly small. Although there is a method for increasing the concentration by eluting into a small amount of solution during the step of column purification after the amplification, when dividing the product of the single-base extension reaction, the sensitivity may be reduced by the division. Therefore, for the template DNA, a single-base extension reaction for a base type X (X is any of the base types A, C, G, and T) may be performed using a selective primer that reacts with the target nucleotide sequence and a terminator (fluorescently labeled terminator) linked with one type of fluorescent dye label. Here, the template DNA may be captured by magnetic beads via a probe, and only the reaction solution containing the product of the single-base extension reaction may be collected. Since the uncollected solution still contains the template DNA, the template DNA is returned to its isolated state by capture with the magnetic beads, and then, similarly to the previous operation, a single-base extension reaction for the next base type is performed using a selective primer that reacts with the target nucleotide sequence and a terminator (fluorescently labeled terminator) linked with one type of fluorescent dye label. By repeating this operation for the number of base types to be detected (for example, base types A, C, G, and T), it is possible to prevent the reduction in the sensitivity when the amount of the template DNA used for the single-base extension reaction is small. Further, even when the amount of the template DNA is not small, this operation is useful for obtaining more stable detection sensitivity because it can remove impurities (such as proteins or DNAs remaining in the solution that may cause noise in fluorescent signals) of the products of the single-base extension reaction.

The method for quantitative analysis of a gene mutation according to the present invention described above can be easily and rapidly carried out by means of a device having the necessary structure, or by means of a kit including the necessary elements.

Accordingly, in another aspect, the present invention provides a device for quantitative analysis of a gene mutation (a quantitative analysis device for analyzing a gene mutation), which comprises:
a measurement unit configured to perform the single-base extension reaction, electrophoresis, and a measurement of a fluorescence signal;
a data analysis unit comprising a measurement data storage part configured to store measurement data obtained by the measurement unit, and a data processing device; and
a control unit,
wherein the measurement unit is configured to perform electrophoresis of a product of the single-base extension reaction for each type of bases to be detected, and measure the fluorescence signal obtained from each electrophoresis, and
wherein the control unit is configured to control the data analysis unit so as to calculate a ratio of a fluorescence intensity of a fluorescent dye in a peak of the product of the single-base extension reaction with respect to a fluorescence intensity of a fluorescent dye in a peak of the internal standard obtained for each base type.

The control unit may further include a reference database that stores previous measurement data.

In such a case, the control unit may be configured to compare the measurement data stored in the measurement data storage part with the previous measurement data stored in the reference database and perform data processing.

The device according to the present invention may further include an output display unit.

In yet another aspect, the present invention provides a kit for quantitative analysis of a gene mutation, comprising:
an internal standard of a constant concentration labeled with a first fluorescent dye;
a primer for a single-base extension reaction for detection of a target nucleotide sequence; and
a terminator for a single-base extension reaction labeled with a second fluorescent dye,
wherein the terminator is comprised in each separate container for each base type so as to be used in the single-base extension reaction for the corresponding base type.

The internal standard, the primers for the single-base extension reaction, and the terminators are as described above. According to the present invention, since the single-base extension reaction is performed for each type of base to be detected, the terminators may be stored in separate containers corresponding to the respective base types so that they can be used independently.

The kit according to the present invention may include, in addition to the above elements, a buffer constituting the reaction solution, enzymes (such as polymerases and reverse transcriptases), calibration standard samples, and the like. By providing, as a kit, the primers, the terminators (the substrates), and the internal standards to be used for the single-base extension reaction, the quantitative analysis of gene mutations can be performed more quickly and easily.

The device and the kit for implementing the method for quantitative analysis of a gene mutation will be described in detail below.

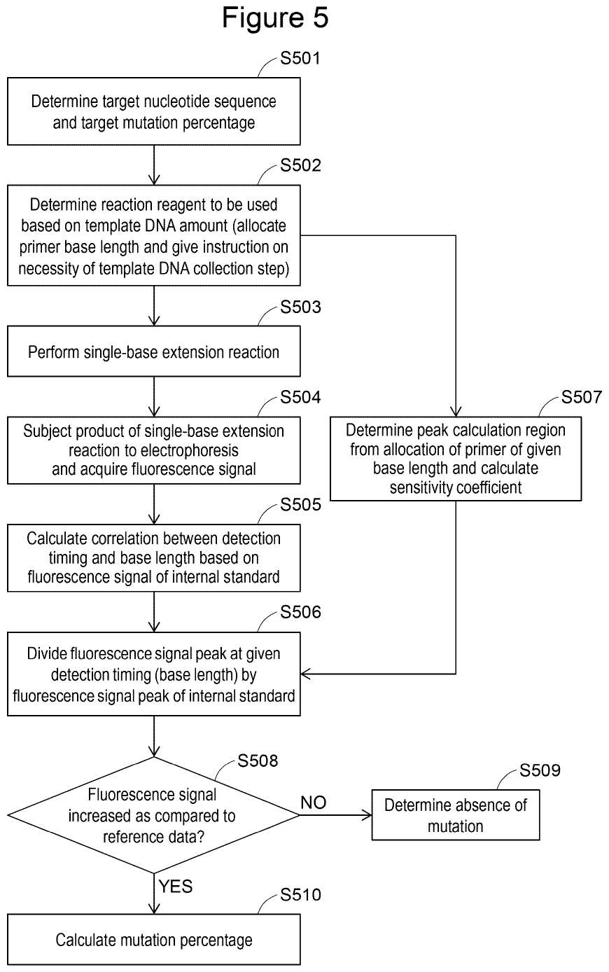

FIG. 5 is a flowchart illustrating one example of the processing procedure in a quantitative analysis device and a quantitative analysis kit for analyzing a gene mutation which exists at a low frequency for implementing the present invention. According to the present invention, a system can be constructed to calculate the mutation percentage (the abundance ratio of the mutant with respect to the wild type) of the target nucleotide sequence over a high dynamic range using the internal standard. First, in step S501, the target nucleotide sequence and the target mutation percentage are determined. Next, in step S502, the reaction reagent to be used is determined based on the amount of the template DNA to be used. In detail, according to the range of the base length to be detected as defined by the specification of the analysis device, the base length of the primer that can be used is assigned, and based on the amount of the template DNA, the protocol (user instructions) indicates whether or not a step of collecting the template DNA is necessary to the user. Subsequently, in step S503, a single-base extension reaction is performed. In step S504, the products of the single-base extension reaction are electrophoresed and fluorescence signals are acquired. At this time, by mixing an internal standard that provides a fluorescence signal at a predetermined detection position (base length), it becomes possible to more accurately determine the relationship between the timing of detection of the fluorescence signal in the electrophoresis and the base length. Step S505 describes the process of calculating the correlation between the detection timing and the base length based on the fluorescence signal of the internal standard (size standard). Next, in step S506, the fluorescence signal peak at the predetermined detection timing (base length) is divided by the fluorescence signal peak of the internal standard so that the mutation percentage can be quantified without the fluorescence signal intensity being saturated even for a sample of a concentration above or equal to a certain concentration. In this case, as shown in step S507, the fluorescence signal peak calculation region is determined based on the assigned primer length corresponding to the predetermined base length based on the information on the reaction reagent used, and the expected sensitivity coefficient K (m, n) is calculated. Then, in step S508, the fluorescence signal of the target is compared with reference data stored in a reference database or with reference data which outputs a baseline value from the reaction reagent settings, to determine whether the fluorescence signal of the target is higher. If the fluorescence signal of the target is not higher than the reference data, “absence of mutation” in the target is determined (step S509). If the fluorescence signal of the target is higher than the reference data, the mutation percentage is calculated in step S510.

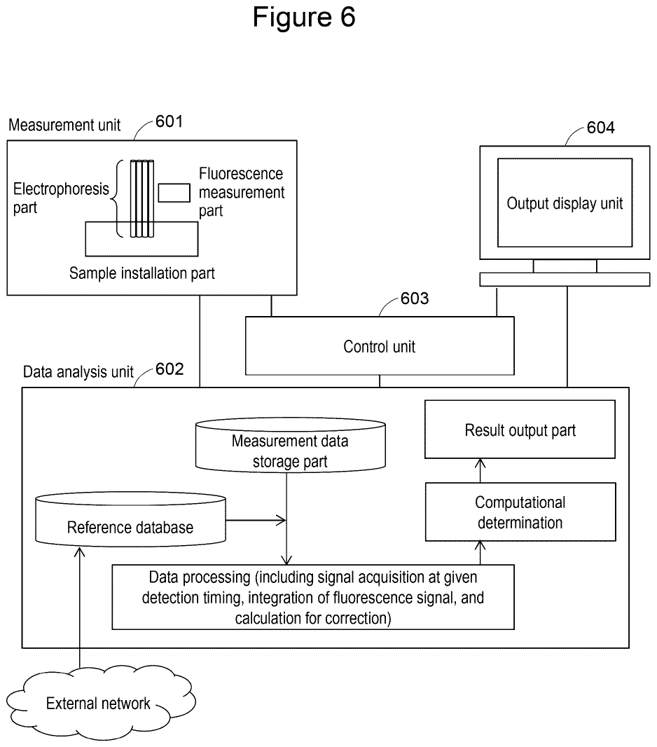

FIG. 6 is a block diagram illustrating one example of a function included in the quantitative analysis device for analyzing a gene mutation according to the present invention. The main components of the device include a measurement unit 601, a data analysis unit 602, a control unit 603, and an output display unit 604. In the measurement unit 601, a sample subjected to a single-base extension reaction is installed in a sample installation part, and the fluorescence signal of the sample flowing through the electrophoresis part over time is measured by the fluorescence measurement part using capillary electrophoresis. The data analysis unit 602 includes a measurement data storage part for storing the measurement data obtained at the measurement unit 601, and a program which implements the data processing can be realized by software. Examples of the data processing include, as shown in the flowchart of FIG. 5, the acquisition of the fluorescence signals at the predetermined detection timing (base length), integration of the fluorescence signals at a specific base length, calculation for correction of a signal drift caused due to longer electrophoresis time, calculation of the ratio of the fluorescence signal intensity in the peak of the product of the single-base extension reaction with respect to the fluorescence signal intensity in the peak of the internal standard obtained in each capillary, quantitative calculation of the abundance ratio of gene mutations, and the like. At this time, it is also possible to use reference electrophoresis data (reference database) stored in advance in the data analysis unit 602. Furthermore, by transmitting and receiving information with an external network, the reference data (reference database) can be updated. It should be noted that the functional control of the measurement unit 601, the data analysis unit 602, and the like can be entirely realized in software by a processor interpretating and executing a program stored in a memory of the control unit 603. Alternatively, some or all of the components, functional units, processing units, processing means, and the like can also be implemented in hardware, for example, by designing them as integrated circuits. Information such as programs, files, and databases for each function can also be stored in a memory, in a storage device such as a hard disk or an SSD (Solid State Drive), or on a recording medium such as an IC card, an SD card, or a DVD. After the data processing, a computational determination is made to determine whether the corrected fluorescence signal after applying the correction value falls within the detectable range of the analysis device, and the like, and the result is outputted via the output display unit 604.

It should be noted that the block diagram shown here represents one example in which the entire system functions as a gene analysis device, and as long as the functions of the measurement unit 601, the data analysis unit 602, the control unit 603, and the output display unit 604 are provided, the gene analysis method of the present invention can be applied.

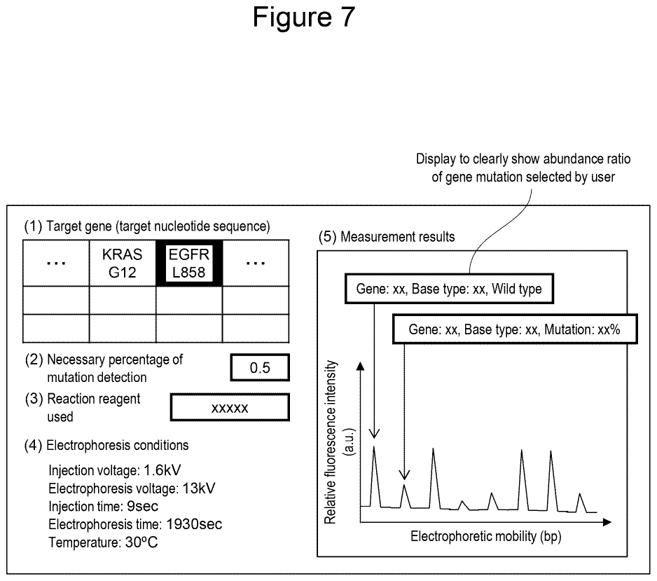

FIG. 7 is a diagram illustrating one example of a user interface screen outputted by the quantitative analysis device for analyzing a gene mutation according to the present invention. As described above with reference to FIG. 5 and FIG. 6, the device allows the user to input the target gene (target nucleotide sequence), the required mutation percentage (mutation detection percentage), and the corresponding reaction reagent to be used. The electrophoresis conditions, such as an injection voltage, injection time, an electrophoresis voltage, electrophoresis time, and a temperature, are inputted in the same manner as the specification provided in a standard capillary electrophoresis device, and in the measurement results, these electrophoresis conditions are displayed as reference data. The electrophoresis measurement results are displayed on the screen, allowing the user to view the signal derived from a mutation in the target gene (target nucleotide sequence) within a specific range of the electrophoretic mobility (bp). For example, even if the data are obtained from electrophoresis respectively performed for the four base types using four capillaries, the measurement result screen displays merged electrophoresis data so that the user can see the abundance ratio of the user-selected gene mutation. This enables the display of detection of a mutation (detection of an SNP) corresponding to a specific base type in the target gene (target nucleotide sequence) and allows calculation and display of the mutation percentage.

The present invention is not limited to the embodiments described above and includes various modifications. For example, the embodiments described above have been explained in detail in order to facilitate the understanding of the present invention, and are not necessarily limited to those including all the described configurations. Further, a part of a configuration of one embodiment can be replaced with a configuration of another embodiment, and it is also possible to add a configuration of one embodiment to a configuration of another embodiment. Moreover, a part of the configuration of each embodiment can be deleted, or supplemented or replaced with other configuration.

### REFERENCE SIGNS LIST

101: Target nucleotide sequence (template DNA)
102: Primers having different base lengths according to target
103: Fluorescent dye
104: Product of single-base extension reaction
201: Polymer solution
202: Capillary
203: Sample injection port
204: Sample (measurement sample)
205: Negative ions other than product of single-base extension reaction
601: Measurement unit
602: Data analysis unit
603: Control unit
604: Output display unit

1. A method for quantitative analysis of a gene mutation, comprising the steps of:
(a) adding an internal standard of a constant concentration labeled with a first fluorescent dye to a test sample;
(b) dividing or preparing the test sample according to the number of types of bases to be detected;
(c) adding a primer for a single-base extension reaction for detection of a target nucleotide sequence to the test sample;
(d) performing a single-base extension reaction using the divided or prepared test sample for each base type, the primer for a single-base extension reaction for detection of a target nucleotide sequence, and a terminator labeled with a second fluorescent dye corresponding to each base type;
(e) subjecting a product of the single-base extension reaction to electrophoresis for each base type; and
(f) measuring mobility in the electrophoresis and a fluorescence intensity of each of the fluorescent dyes, and detecting a wild type and a mutant of the target nucleotide sequence based on the fluorescence intensity,
wherein a ratio of the fluorescence intensity of the second fluorescent dye in a peak of the product of the single-base extension reaction with respect to the fluorescence intensity of the first fluorescent dye in a peak of the internal standard obtained for each base type is calculated, and based on the ratio, a quantitative ratio of the product of the single-base extension reaction for each base type is quantified.

2. The method according to claim 1, wherein the steps are carried out in the order of the steps (a), (b), (c), (d), (e), and (f).

3. The method according to claim 1, wherein the steps are carried out in the order of the steps (c), (a), (b), (d), (e), and (f).

4. The method according to claim 1, wherein in the step (d), the single-base extension reaction is performed in the presence of, in addition to the terminator labeled with a second fluorescent dye corresponding to each base type, a terminator unlabeled with a fluorescent dye corresponding to a base type different from the base type.

5. The method according to claim 1, wherein in the step (a), the internal standard of a constant concentration is a product of a single-base extension reaction of a known concentration extended by a base of the same base type as a base to be detected, and
wherein the ratio of the fluorescence intensity of the second fluorescent dye in the peak of the product of the single-base extension reaction with respect to the fluorescence intensity of the first fluorescent dye in the peak of the product of the single-base extension reaction of the known concentration is calculated, and based on the ratio, the quantitative ratio of the product of the single-base extension reaction for each base type is quantified.

6. The method according to claim 1, further comprising, after performing the steps (c) and (d), a step of:
(g) capturing the test sample that serves as a template for the single-base extension reaction using a solid phase for capturing the test sample, and collecting a reaction solution comprising the product of the single-base extension reaction,
wherein the steps (c), (d), and (g) are repeatedly carried out the number of times corresponding to the number of types of bases to be detected using the same test sample to obtain, as the step (b), a product of a single-base extension reaction prepared for each type of base to be detected corresponding to the test sample, and thereafter the steps (a), (e), and (f) are carried out.

7. The method according to claim 6, wherein the solid phase is magnetic beads.

8. The method according to claim 1, wherein in the step (b), the test sample is divided into two, three, or four portions.

9. The method according to claim 1, wherein the gene mutation comprises a single nucleotide polymorphism (SNP).

10. The method according to claim 1, wherein the electrophoresis is capillary electrophoresis.

11. A device for quantitative analysis of a gene mutation for implementing the method according to claim 1, comprising:
a measurement unit configured to perform a single-base extension reaction, electrophoresis, and a measurement of a fluorescence signal;
a data analysis unit comprising a measurement data storage part configured to store measurement data obtained by the measurement unit, and a data processing device; and
a control unit,
wherein the measurement unit is configured to perform electrophoresis of a product of the single-base extension reaction for each type of bases to be detected, and measure the fluorescence signal obtained from each electrophoresis, and
wherein the control unit is configured to control the data analysis unit so as to calculate a ratio of a fluorescence intensity of a fluorescent dye in a peak of the product of the single-base extension reaction with respect to a fluorescence intensity of a fluorescent dye in a peak of an internal standard obtained for each base type.

12. A kit for quantitative analysis of a gene mutation for implementing the method according to claim 1, comprising:
an internal standard of a constant concentration labeled with a first fluorescent dye;
a primer for a single-base extension reaction for detection of a target nucleotide sequence; and
a terminator for a single-base extension reaction labeled with a second fluorescent dye,
wherein the terminator is comprised in each separate container for each base type so as to be used in the single-base extension reaction for the corresponding base type.
